(19) United States
(12) Patent Application Publication
(10) Pub. No.: US 2003/0035779 A1
(43) Pub. Date: Feb. 20, 2003

(54) Biofilm therapy process and elements

(76) Inventors: Dale Brown, Wharton, TX (US); Robert D. White, Chadds Ford, PA (US)

Correspondence Address: BANNER & WITCOFF, LTD., 28 STATE STREET, 28th FLOOR, BOSTON, MA, 02109, US

(21) Appl. No.: 10/005,902

(22) Filed: Dec. 4, 2001

Related U.S. Application Data
(60) Provisional application No. 60/254,457, filed on Dec. 8, 2000.

(51) Int. Cl. A61K 7/16

(52) U.S. Cl. 424/49

(57) ABSTRACT

Disclosed is a self-treatment process and system for periodontal patients with gingival detachment of about 3 mm and greater comprising regularly removing biofilms from various supragingival, interproximal and subgingival surfaces, wherein “soft abrasives” are physically worked into the biofilms using toothbrushing, proxy brushing and flossing. The process is also used to help control those inflammation related substances resulting from gum disease that are associated with heart disease.

### PRIORITY CLAIM

This application claims domestic priority from copending U.S. Provisional Application Serial No. 60/254,457, filed Dec. 8, 2000, the disclosure of which is hereby incorporated herein by reference.

### BACKGROUND OF THE INVENTION

Current “at-home” oral care practice in the U.S. is at least partly responsible for 13 million adults being treated annually for gum disease, as well as for the 67 million adults indicating some periodontal disease, i.e., gingival detachment of at least 3 mm. Emphasis on toothbrushing with fluoride, whitening toothpastes and rinsing with germ fighting rinses fails to address the fundamental oral care problem of those patients suffering from gum disease, i.e., the need to physically remove supragingival, interproximal and subgingival plaque from critical tooth surfaces . . . daily.

Aged plaque is now described as a biofilm. Biofilms below the gumline and between teeth are recognized as the host for those pathogens responsible for gum disease, as well as C-reactive protein which is identified with heart disease. Throughout nature, biofilms have a reputation for being notoriously difficult to remove. Biofilms are unique ecosystems that are most pervasive; they extend from “slimes” common to various industrial processes to inflammation in humans and animals.

Unfortunately, as our understanding of gum disease expands and the influence gum disease has on other chronic diseases such as heart disease is better understood, the oral hygiene response to this overall enlightened understanding has, to date, been at best, ineffective. The size of the U.S. periodontal market confirms this. For example, the target for the oral hygiene of periodontal patients and periodontal patients with indications of heart disease is unequivocally the ongoing control of supragingival, interproximal and subgingival biofilms. The nature of these biofilms and their respective location on the teeth of periodontal patients requires a system or process for routinely physically removing and/or disrupting these biofilms. This process calls for multiple devices suitable for physically removing and/or disrupting these various biofilms regularly. Preferably, this removal and/or disruption is effected at least daily, and more preferably, several times daily and at least after each meal. The present invention is directed to such a process.

### OBJECTS OF THE INVENTION

An object of the invention is to provide periodontal patients with a self-treatment process that physically removes biofilms from all tooth surfaces.

Another object of the invention is to provide a process to physically remove and/or disrupt biofilms on various tooth surfaces of periodontal patients regularly, and thereby help inflammation related substances associated with heart disease.

A further object of the invention is to provide a self-treatment process for periodontal patients that stabilizes and/or improves gingival detachment.

Yet another object of the invention is to provide a process for treating gum disease of patients who are at-risk of other chronic diseases.

Still, another object of the invention is to provide devices suitable for use in a process to control biofilms on tooth surfaces of periodontal patients.

Yet another object of the invention is to help control inflammation related substances in periodontal patients with a propensity to develop heart disease.

### SUMMARY OF THE INVENTION

The present invention is directed to a patient self-treatment process for periodontal patients with gingival detachment of 3 mm or greater comprising physically removing biofilms from various tooth surfaces periodically, using soft abrasives physically worked into said biofilms with various devices. Specifically, these devices can include: toothbrushes and proxy brushes with ribbed and grooved bristles and interproximal devices that release soft abrasives onto supragingival, interproximal and subgingival biofilms. The process includes working these soft abrasives onto these biofilms while the devices are being worked supragingivally, interproximally and subgingivally.

### BRIEF DESCRIPTION OF THE DRAWINGS

FIGS. 1 through 4 are perspective views of several biofilm therapy treatment toothbrushes with preferred handle shapes. Each toothbrush head is fitted with ribbed and grooved bristles.

FIGS. 5 and 6 are perspective views of proxy brushes fitted with ribbed and grooved bristles.

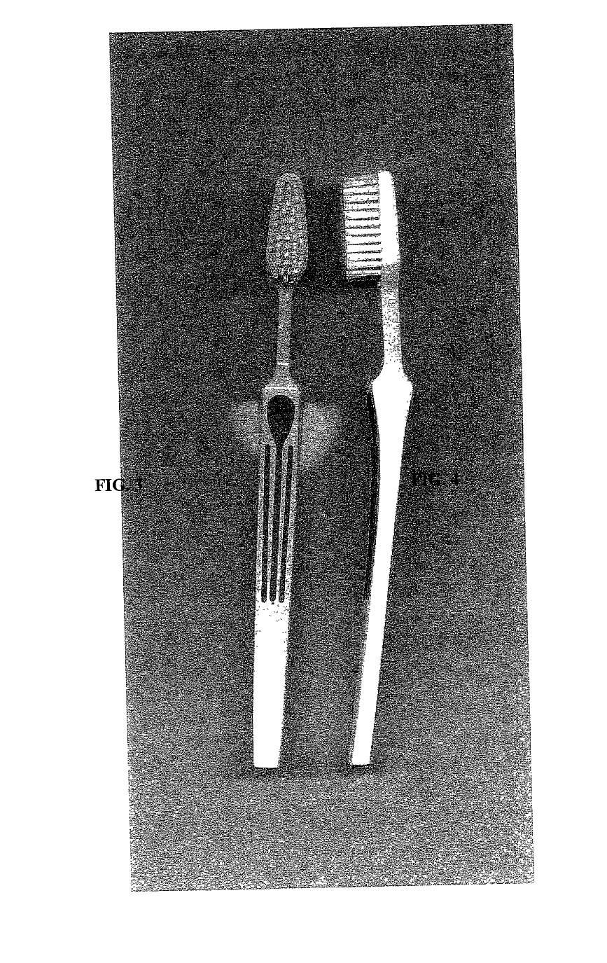

FIGS. 7 and 8 are perspective views of ribbed and grooved bristles.

FIG. 9 (also referred to as Graph 1) summarizes the biofilm removal clinical data for a ribbed and grooved bristle toothbrush compared with various round bristled toothbrushes.

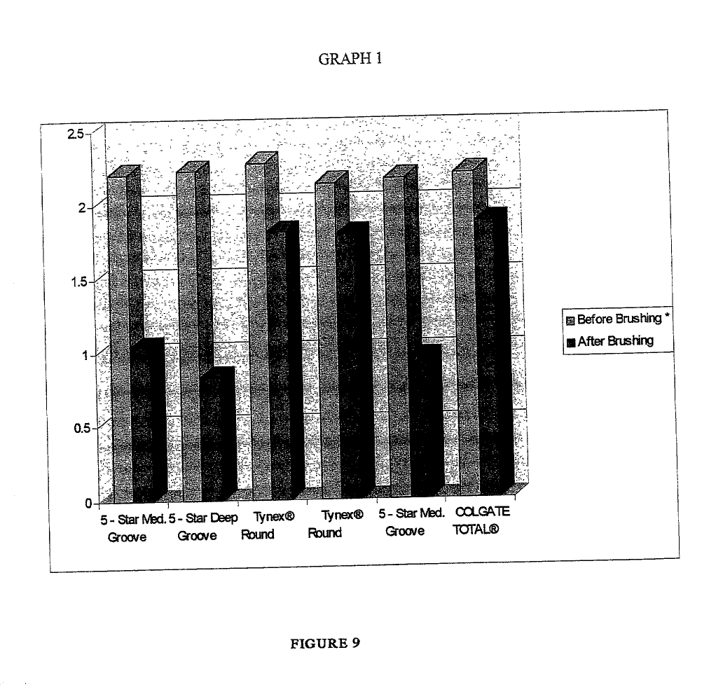

### DETAILED DESCRIPTION OF THE PREFERRED EMBODIMENTS

It is estimated that there are approximately 13 million patients in the U.S. receiving periodontal treatment from periodontists with approximately 67 million adults over the age of 40 indicating some gingival detachment of 3 mm or greater.

The biofilm therapy process of the present invention is marketed under the trademark, Biofilm Therapy™. It provides daily physical removal of biofilms from critical tooth surfaces for periodontal patients undergoing professional treatment including: planing, scaling, etc., as well as patients undergoing prescription treatments including Atridox®, PerioChip®, Periostat®, doxycycline, minocycline, tetracycline, metronidazole, etc.

The physical removal of biofilm must be carried out regularly by: periodontal patients, those with gingival detachment of 3 mm or greater, and chronic periodontal sufferers; in order to maximize their professional treatment and/or to stabilize or reverse their level of gingival detachment.

For those periodontal patients suffering from other chronic diseases, such as heart disease, diabetes, osteoporosis, etc., there is an added incentive to comply with the biofilm therapy treatment of the present invention. That is, gum disease has now been linked with exacerbating and/or complicating these other chronic conditions. Specifically, gum disease contributes to increased levels of inflammation based substances associated with prospective heart patients. For these “at-risk” periodontal patients regular physical removal of biofilms from critical tooth surfaces could be as critical to their continuing health as taking medication for their chronic condition(s).

Daily physical removal of biofilms is the sole responsibility of the individual. The biofilm therapy treatment of the present invention provides an individual with a self-administered, soft-tissue management program that physically removes biofilms from: (a) supragingival, (b) interproximal, as well as (c) subgingival tooth surfaces.

The biofilm therapy treatment process of the present invention includes three key elements:

1. physically removing biofilm from supragingival tooth surfaces. This can be accomplished by using a ribbed and grooved bristled biofilm therapy toothbrush in combination with a complimentary soft abrasive toothpaste that is responsive to the ribbed and grooved bristles of the toothbrush and thereby removes more biofilm than traditional round bristle/toothpaste combination. As shown in Table 1 and Graph 1 below, the biofilm therapy toothbrush/soft abrasive toothpaste combination of the invention removes up to 25% more supragingival plaque (or supragingival biofilm) than traditional round bristle toothbrush/toothpaste combinations. However, physical removal can also be accomplished with slightly less effectiveness using traditional round bristles;

2. physically removing biofilm from interproximal tooth surfaces. This can be accomplished by using a ribbed and grooved bristle biofilm therapy proxy brush in combination with a complimentary soft abrasive containing proxy gel that is responsive to the ribbed and grooved bristles of the proxy brush. The combination thereby removes more interproximal biofilm than a traditional proxy brush used alone and reaches interproximal surfaces not reached by toothbrushing or rinsing. However, physical removal can also be accomplished with slightly less effectiveness using traditional round bristles; and

3. physically removing biofilm from interproximal and subgingival surfaces.

This can be accomplished by using a dental floss or dental tape containing a substantial quantity of releasable “soft abrasive” that, once released during flossing, can be worked onto interproximal and subgingival biofilms, found at those interproximal surfaces and not reached by toothbrushing, rinsing and/or proxy brushing.

The three elements of the biofilm therapy process of the present invention can be carried out in any sequence. Generally each element is carried out at least once every 24 hours and preferably at least twice daily. Ideally, each element is carried out after every meal or snack. Some elements may be carried out more frequently than others. At least two of the elements in the process are to be carried out periodically.

Surprisingly, periodontal patients who generally have a history of not regularly using interproximal devices, let alone multiple interproximal devices, have been observed to routinely perform the three elements of the biofilm therapy process of the present invention at least once daily. Many carry out all three elements several times throughout the day. Most of these “high compliance” periodontal patients have at least one or more chronic conditions and appear to be motivated by more than the risk of potential tooth loss. That is, the risk of exacerbating and/or complicating other chronic conditions for which they are presently taking medication seems to be a genuine driving force behind the extraordinary compliance. This is particularly true in the case of heart disease patients, where it is clear that controlling biofilm levels helps control levels of inflammation based substances associated with heart disease.

A second major contributor to the high compliance reported for the biofilm therapy process, with its three distinct elements, appears to be the targeted biofilm itself. That is, the flossing, proxy brushing, serious toothbrushing all with “soft abrasives” are focused on physically removing biofilms. It is now abundantly clear that biofilms host the pathogens that cause gum disease. Working devices regularly between teeth and subgingivally, etc., to remove biofilms, as called for by the process of the present invention, seems to make sense to “at-risk” adults. In contrast, “flossing” regularly without a specific target obviously heretofore has failed to interest approximately 87% of all adults, who are classified ass “non-flossers”. This proxy brushing, toothbrushing and flossing with a specific purpose in mind, i.e., controlling pathogen and/or inflammation based substances associated with heart disease appears to be fundamental to high compliance.

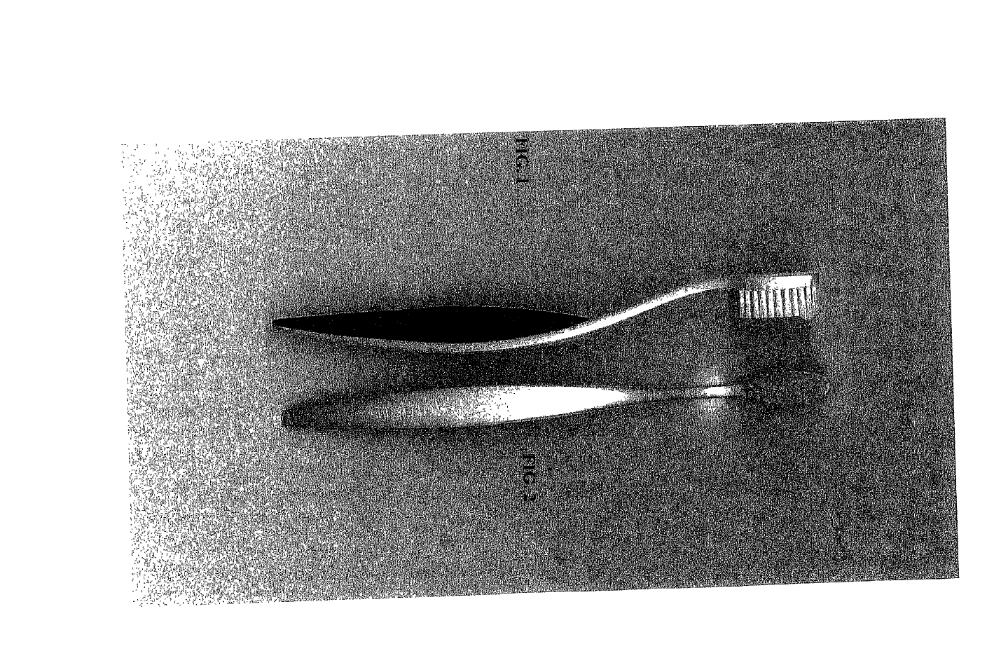

Preferred biofilm therapy treatment toothbrushes suitable for the purposes of the present invention are described and claimed in U.S. patents to Schiff and Hill, U.S. Pat. Nos. 5,993,784; 6,086,373 and 6,138,314 and pending application Ser. No. 09/189,196. Particularly preferred biofilm therapy treatment toothbrushes are the 5-Star bristled toothbrushes claimed and described by Schiff and Hill, fitted with the triangular shaped Jordan handle shown in FIGS. 1 and 2 of the drawings or the angled handle shown in FIGS. 3 and 4.

Suitable “soft abrasive” toothpastes are described in detail in Table 2, Examples 1 through 8 and 28. The soft abrasives in these toothpastes compliment the ribbed and grooved bristles and thereby physically remove supragingival biofilm more efficiently than round bristle/traditional toothpaste combinations.

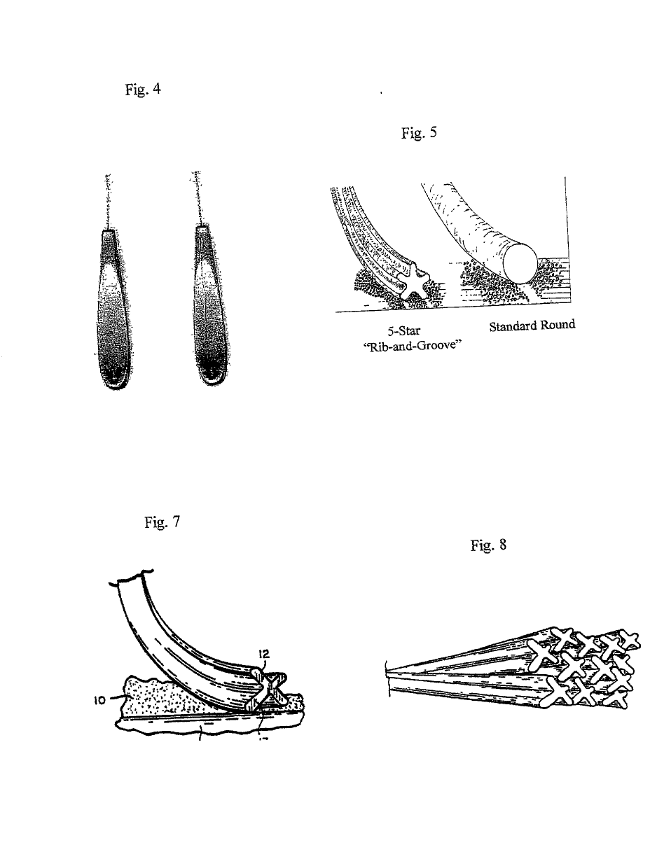

The bristles for preferred biofilm therapy treatment proxy brushes suitable for the purposes of the present invention are described and claimed in U.S. Pat. Nos. 5,993,784; 6,086,373 and 6,138,314. Particularly preferred bristles for these proxy brushes are five ribbed bristles as shown in FIGS. 7 and 8. Three ribbed bristles are also preferred.

Suitable “soft abrasive” proxy gels include those described and claimed in copending Hill patent application, Ser. No. 60/227,255, which is incorporated herein by reference. The “soft abrasives” in these proxy gels compliment the ribbed and grooved bristles in the proxy brush and thereby present more soft abrasives to the biofilm than round bristled proxy brushes. Examples of suitable proxy gels with soft abrasive are described in Table 3 and in Examples 9 through 13.

Preferred biofilm therapy treatment dental flosses and tapes suitable for the process of the present invention are described and claimed in various U.S. patents and patent applications as follows:

Dental flosses described and claimed in U.S. Pat. Nos. 4,911,927; 5,098,711; 5,165,913; 5,665,374; and 5,711,935.

Dental tapes as described in copending applications, Ser. Nos. 60/227,196; 60/227,239; 60/227,240; 60/227,244; 60/227,255; 60/227,433; 60/227,246 and 60/227,242, the disclosures of which are hereby incorporated herein by reference.

All of the foregoing interproximal devices are loaded or coated with substantial quantities of soft abrasives as described in Tables 4 and 5. These soft abrasives are released in total or in part and thereafter worked into the interproximal and subgingival biofilms by the floss or tape, thereby assisting in the disruption and/or removal of said biofilms.

For the purposes of the present invention, the following definitions apply to the various terms used throughout this specification:

Biofilm Therapy is a process for removing biofilms from various tooth surfaces using various devices in combination with various products suitable for physically disrupting and/or removing biofilms from tooth surfaces: supragingivally, interproximally and subgingivally. (Biofilm Therapy™ is a trademark of the Perident/Hill & White Joint Venture.)

Periodontal patients include those patients with gingival detachment of about 3 mm or greater and include periodontal patients under professional treatment, chronic sufferers of periodontal disease. Most periodontal patients either have one or more other chronic diseases or a propensity to succumb to one or more other chronic diseases.

“At-Risk” Patients are those periodontal patients with another chronic disease that is complicated and/or exacerbated by periodontal disease.

Dental Devices include all devices that can be worked with various substances to physically remove and/or disrupt biofilms. These include: toothbrushes, proxy brushes, dental flosses and dental tapes. Particularly preferred devices for the process of the present invention include: ribbed and grooved toothbrushes and proxy brushes and loaded or coated dental flosses and dental tapes.

Therapeutic Substances include those active ingredients that affect periodontal disease or that can control the inflammation related substances associated with heart disease.

Soft Abrasives are those inert substances that, when physically worked onto biofilms by the various dental devices of the process of the invention, physically remove and/or disrupt biofilms without damaging the hydroxy appetite of the tooth surfaces. These include: abrasives synthetic and natural, and whitening and tartar control ingredients including peroxides, baking soda, silica, alumina silicates, tricrasyl phosphate, tetrasodium pyrophosphate. Particle sizes and more details on these substances are provided in the Tables and Examples detailed below.

Biofilms include the various “plaque based” coatings that continually form on tooth surfaces and, when not removed physically, have the propensity to host pathogens that cause gum disease. Other unsavory substances are also associated with biofilms include various substances related to inflammation that are associated with heart disease and anaerobic bacteria.

ULTRAMULSIONS® and MICRODENT® are emulsions of nonionic surfactants and polydimethylsiloxanes as described in U.S. Pat. Nos. 4,950,479; 5,032,387; 5,538,667; 5,645,841 and 5,561,959, respectively.

Viscosity control agents include various natural and synthetic thickeners and gelling agents suitable use in Proxy Gels, including: carboxymethyl cellulose, gum tragacanth, methyl cellulose, etc.

Mouth conditioners include: ULTRAMULSIONS®, MICRODENT® and viscosity control agents.

A ribbed and grooved toothbrush was compared with various round bristle toothbrushes in a clinical study to establish comparative plaque removal of the two bristle types. Results are set out in Table 1 and Graph 1.

TABLE 1
Toothbrush Clinical Summary of Mean Plaque Scores

| | | Before | After | t-Test |
|---|---|---|---|---|
| Diameter | Bristle | Brushing* | Brushing | vs. Round |
| 0,007 | 5-Star Med. Groove | 2.21 | 1.04 | P < 0.0001 |
| 0.007 | 5-Star Deep Groove | 2.23 | 0.83 | P < 0.0001 |
| 0.007 | Tynex ® Round | 2.28 | 1.81 | — |
| 0.007 | Tynex ® Round | 2.14 | 1.79 | — |
| 0.007 | 5-Star Med. Groove | 2.17 | 0.95 | P < 0.0001 |
| Medium | Colgate Total ® | 2.2 | 1.87 | No sig. Duff. |

*ANOVA no sig. diff.

Examples 1 to 26 as detailed in Tables 2 through 5 are illustrative of the various element of the invention.

TABLE 2
Soft Abrasive Dentifrices

| | | | | Soft |
|---|---|---|---|---|
| | | Soft | | Abrasive |
| Example | | Abrasive | Plaque Fighting | Particle Size |
| No. | Type | % by wt. | Ingredient | (in microns) |
| 1 | Gel | 30.0 silica | ULTRAMULSION ®-10 | 5-25 |
| 2 | Gel | 25.0 silica | ULTRAMULSION ®-10 | 30-60 |
| 3 | Gel | 27.0 silica | Triclosan | 5-25 |
| 4 | Gel | 32.0 silica | Chlorhexidine | 5-25 |
| 5 | Paste | 49.0 DCP | ULTRAMULSION ®-10 | 5-25 |
| 6 | Paste | 43.0 DCP | ULTRAMULSION ®-10 | 30-60 |
| 7 | Paste | 40.0 | Triclosan | 5-25 |
| | | aluminum | | |
| | | silicate | | |
| 8 | Paste | 43.0 DCP | Eucalyptol | 5-25 |
| | | | Menthol | |
| | | | Thymol | |
| | | | Methyl salicylate | |

TABLE 3
Soft Abrasive Proxy Gels

| Example | Mouth Conditioners | Viscosity Control | Soft Abrasive |
|---|---|---|---|
| No. | % by wt. | Agent | % by wt. |
| 9 | ULTRAMULSION ®- | CMC | Sident-10, 0.5 |
| | 10, 5.0 | | |
| 10 | Xanthan Gum, 0.1 | Poloxamer 407 | Sident-22, 1.0 |
| 11 | CMC, 0.5 | Xanthan Gum | Zeodent 113, 1.5 |
| 12 | ULTRAMULSION ®- | Xanthan Gum | dicresl |
| | 10, 2.0 | | phosphate, 2.0 |
| 13 | Carrageenin, 0.1 | Poloxamer 407 | silica, 2.0 |

TABLE 4
Soft Abrasive Multifilament Dental Floss

| | | | Soft Abrasive | | Total |
|---|---|---|---|---|---|
| Example | | | Type | | Load |
| No. | Floss Type | Denier | % by wt. | Conditioners | in mg/yd |
| 14 | Textured nylon | 800 | DCP | ULTRAMULSION ®-10 | 90 |
| 15 | Textured nylon | 700 | DCP | ULTRAMULSION ®-10 | 75 |
| 16 | Textured nylon | 840 | TSPP | ULTRAMULSION ®-10 | 95 |
| 17 | Textured nylon | 840 | TSPP | Poloxamer 407 | 95 |
| 18 | Non-texturized | 700 | DCP | PEG-1450 | 70 |
| | polypropylene | | | | |
| 19 | Non-texturized nylon | 840 | DCP | ULTRAMULSION ®-10 | 90 |

Note: Aspirin, other NSAIDS or salicylic acid could be included in the load of Examples 14, 15, and 19 at a range from between about 0.5% by weight and 2% by weight to control inflammation related substances associated with heart disease.

TABLE 5
Soft Abrasive Monofilament Dental Tape

| | | Soft | | |
|---|---|---|---|---|
| Ex- | | Abrasive | | Coating |
| ample | | Type % | | Wt. in |
| No. | Floss Type | by wt. | Conditioners | mg/yd |
| 20 | Narrow tape | TSPP, 8 | ULTRAMULSION ®-10 | 40 |
| | Fibaclean | | | |
| 21 | Wide tape | TSPP, 14 | ULTRAMULSION ®-10 | 70 |
| | Fibaclean | | | |
| 22 | Narrow tape | DCP, 6 | Poloxamer 407 | 35 |
| | Fibaclean | | | |
| 23 | Wide Tape | DCP, 6 | PEG-1450 | 60 |
| | Polypropylene | | | |
| 24 | Narrow Tape | Silica, 4 | ULTRAMULSION ®-20 | 40 |
| | Polypropylene | | | |
| 25 | Wide Tape | Silica, 4 | Poloxamer 407 | 55 |
| | Fibaclean | | | |
| 26 | PTFE | Silica, 4 | ULTRAMULSION ®-10 | 30 |

### EXAMPLE 27

A soft abrasive containing proxy gel suitable for use with a ribbed and grooved bristle proxy brush was prepared having the following formula:

| Ingredient | % weight/weight |
|---|---|
| Water (deionized) | 6.20 |
| Ultramulsion ®-35/2.5 mmcs (20% sol) | 7.50 |
| Flavor | 0.40 |
| Sorbitol 70% | 45.14 |
| Disodium EDTA | 0.05 |
| Cetyl pyridinium chloride | 0.06 |
| Ethanol | 18.00 |
| Sident-10 | 2.0 |
| Sodium saccharin | 0.15 |
| Carboxymethylcellulose | 0.50 |
| Glycerin | 20.00 |

The foregoing formulation was prepared as follows:

Previously prepared ULTRAMULSION®-35/2.5 mmcs@20%) was added to water followed by the flavor with mixing. With stirring the following were added to the mixture sequentially: Sorbitol (70%), EDTA, CPC. Ethanol was then added, followed by saccharin with stirring until dissolved. A premix of glycerin and CMC (lump-free) was added slowly with stirring until homogenous. Mixture de-aerated with vacuum to remove bubbles and transferred into tubes.

### EXAMPLE 28

A soft abrasive toothpaste was prepared having the following formula:

| I. | Ingredient | % weight/weight | |
|---|---|---|---|
| 1. | Deionized water | 32.14 | |
| 2. | Sorbitol 70% Aq. | 19.00 | |
| 3. | TetraPotassium pyrophosphate | 3.90 | |
| 4. | Sodium saccharin | 0.20 | |
| 5. | Ultramulsion ®-10 | 2.00 | Pre-mix #2 |
| 6. | PEG-12 | 1.00 | |
| 7. | Sident-9 | 14.00 | |
| 8. | Sident-22a | 6.00 | |
| 9. | Aluminum oxide | 10.00 | |
| 10. | Titanium dioxide | 0.40 | |
| 11. | Vanillamint flavor | 1.00 | |
| 12. | Sodium monofluorophosphate | 0.76 | |
| 13. | Cellulose Gum-7-MF | 0.80 | Pre-mix #1 |
| 14. | Glycerine-USP | 8.00 | Pre-mix #1 |
| 16. | Sodium Lauryl Sulfate | 0.80 | |
| | | 100.00 | |

Pre-Mix Instructions:

A. Pre-mix #1: Slurry the Cellulose Gum into the Glycerine, insure the mix is lump-free (no fish-eyes).

B. Pre-mix #2: Prepare a 20% aqueous dispersion: 2% ULTRAMULSION® and 8% Deionized Water.

II. Mixing Instructions:

A. To the DI Water, add ingredients 2 thru 6 in order, with moderate agitation. Insure all ingredients are completely dissolved before proceeding.

B. When A is complete, add Sident-9 and Sident22s. Completely disperse before proceeding.

C. Add the Aluminum Oxide and Titanium Dioxide; completely disperse.

D. Add Flavor and Sodium MFP; completely disperse.

E. Add Pre-mix #1 and SLS. Mix and deaerate entire batch until uniformly smooth.

Finished Product Specifications:

| | |
|---|---|
| Specific Gravity | 1.45-1.55 |
| pH-I:10 dilution | 8.2-8.7 |
| % Na MFP-w/w | 0.7-0.8 |

The present invention has been described in detail, including the preferred embodiments thereof. However, it will be appreciated that those skilled in the art, upon consideration of the present disclosure, may make modifications and/or improvements on this invention and still be within the scope and spirit of this invention as set forth in the following claims.

What is claimed is:

1. A self-treatment process for periodontal patients with gingival detachment of about 3 mm and greater, comprising regularly physically removing biofilms from supragingival, interproximal and subgingival tooth surfaces by sequentially administering soft abrasives into said biofilms using toothbrushing, proxy brushing and flossing devices.

2. A self-treatment process for periodontal patients with gingival detachment of about 3 mm and greater and with a risk of heart disease, comprising regularly physically removing biofilms from supragingival, interproximal and subgingival tooth surfaces and simultaneously controlling inflammation related substances associated with heart disease by sequentially administering soft abrasives onto:
a. supragingival tooth surfaces with toothbrushing,
b. interproximal tooth surfaces with proxy brushing, and
c. interproximal and subgingival surfaces with flossing.

3. The process of claim 2, wherein:
a. toothbrushing is carried out with a ribbed and grooved toothbrush in combination with a soft abrasive containing toothpaste, wherein the combination physically works soft abrasive into supragingival plaque,
b. proxy brushing interproximal spaces not reached with toothbrushing is carried out with a ribbed and grooved bristled proxy brush in combination with a soft abrasive containing proxy gel, wherein the combination physically works soft abrasive into interproximal biofilms, and
c. flossing subgingival and interproximal spaces not reached by toothbrushing and proxy brushing is carried out with an interproximal and subgingival flossing device containing releasable soft abrasive, wherein said device physically works released soft abrasives into interproximal and subgingival biofilms.

4. The process of claim 3, wherein said proxy gel contains a therapeutic substance selected from the group consisting of fluoride, cetyl pyridinium chloride, triclosan, chlorhexidine digluconate, metronidazole, doxycycline, tetracycline, minocycline and mixtures thereof.

5. The process of claim 3, wherein said flossing device contains a releasable therapeutic substance selected from the group consisting of fluoride, cetyl pyridinium chloride, triclosan, chlorhexidine digluconate, metronidazole, doxycycline, tetracycline, minocycline and mixtures thereof.

6. The process of claim 3, wherein said flossing device is selected from the group consisting of multifilament floss, texturized multifilament floss, PTFE tape and elastomeric tape.

7. The process of claim 2, wherein said proxy gel and said flossing device each contain an inflammation related substance associated with heart disease.

8. A system adapted for self-administering soft abrasive onto biofilms on the supragingival, interproximal and subgingival tooth surfaces of periodontal patients with gingival detachment of at least about 3 mm, said system comprising two or more devices selected from the group consisting of toothbrushes, proxy brushes and flossing devices and combinations thereof.
